(19) United States
(12) Patent Application Publication
(10) Pub. No.: US 2004/0219662 A1
(43) Pub. Date: Nov. 4, 2004

(54) Analytical and diagnostic instrument

(76) Inventor: Wolfgang Geiger, Ansbach (DE)

Correspondence Address: HARNESS, DICKEY & PIERCE, P.L.C., P.O.BOX 8910, RESTON, VA, 20195, US

(21) Appl. No.: 10/777,687

(22) Filed: Feb. 13, 2004

(30) Foreign Application Priority Data
Feb. 13, 2003 (DE) 10306018.9

(51) Int. Cl. C12M 1/34

(52) U.S. Cl. 435/287.1; 435/288.5

(57) ABSTRACT

An analytical and diagnostic instrument includes an analysis chip for testing biological material. The analysis chip includes a carrier and biosensor, and an inlet opening and outlet opening for a disinfection fluid. The analytical and diagnostic instrument also includes a disinfection device provided for applying the disinfection fluid to the analysis chip.

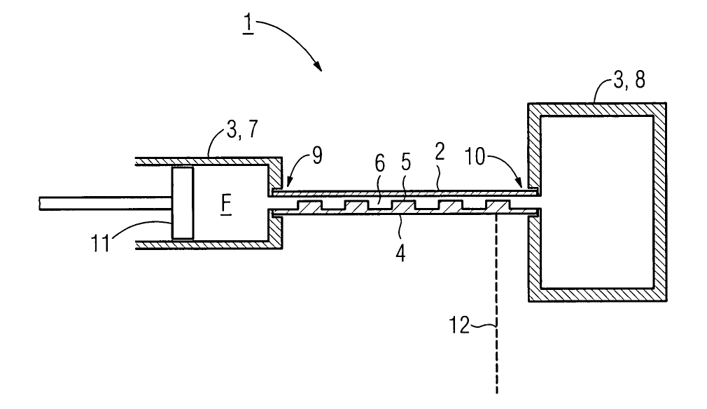

The present application hereby claims priority under 35 U.S.C. §119 on German patent application number DE 103 06 018.9 filed Feb. 13, 2003, the entire contents of which are hereby incorporated herein by reference.

### FIELD OF THE INVENTION

The invention generally relates to an analytical and diagnostic instrument for testing biological samples.

### BACKGROUND OF THE INVENTION

An analysis chip for testing biological samples is known from DE 198 19 537 A1. This analysis chip has a carrier which is produced by micro-injection molding of plastic and on which a biomolecular matrix is applied. A biological sample to be tested flows through a meandering structure inside the analysis chip.

In the future, it is expected that analysis chips will be increasingly used in the self-test market, for example for testing blood. In this connection, there is a considerable risk of the analysis chip being contaminated by the test sample and yet being discarded in household waste, despite the fact that it should be disposed of as infectious waste.

In the professional sector, that is to say involving physicians or hospitals, used test materials which may possibly be contaminated, for example analysis chips, are in principle to be disposed of as infectious waste (biohazardous waste). The cost associated with this greatly exceeds the cost of disposing of waste that does not require monitoring.

### SUMMARY OF THE INVENTION

An object of an embodiment of the invention is to reduce or even minimize the risks associated with using analysis chips for testing biological material, particularly in the self-test sector. A further aim is to make disposal of used analysis chips, particularly in the professional sector, more economic.

According to an embodiment of the invention, an object may be achieved by an analytical and diagnostic instrument.

An analytical and diagnostic instrument may include, on the one hand, an analysis chip, and, on the other hand, a disinfection device. The analysis chip may contain a carrier, at least one biosensor, and an inlet opening and an outlet opening for a disinfection fluid. A storage vessel and collecting vessel for the disinfection fluid may be connected or may be connected to the inlet opening and outlet opening, respectively, for the disinfection fluid. The disinfection fluid is preferably liquid, but it can also be gaseous. Likewise, a number of starting materials may form a disinfection agent only by reaction in the analysis chip.

In any case, the disinfection fluid can reach the possibly contaminated areas of the analysis chip in the disinfection process. Depending on the configuration of the analysis chip, this may be achieved by the fact that it is completely wetted by the disinfection fluid, or at least its area exposed to the biological test sample is wetted by the disinfection fluid. The disinfection fluid flows through a fluid system of the analysis chip, for example by gravity, by capillary forces, or by forces applied from outside.

The fluid system of the analysis chip can, for example, include channels, in particular meandering channels, and reaction chambers. The disinfection fluid can be brought into contact with the analysis chip, and the disinfection fluid can be mixed with the previously tested biological material, by way of agitation, osmosis or diffusion.

To store the unused disinfection fluid, on the one hand, and to collect the used disinfection fluid to which the tested biological material has been added, on the other hand, it is possible to provide either separate vessels or just one single vessel. The last-mentioned option is particularly expedient when the biological material to be tested does not have to flow through an elongate channel inside the analysis chip, but instead reaches the biosensor or biosensors, for example in the form of dots on the surface of the carrier of the analysis chip, by a short, direct route.

According to a first preferred embodiment, the analytical and diagnostic instrument includes the disinfection device as an integral component part which is permanently connected to the analysis chip or can easily be connected to it, for example plugged onto it. The disinfection device is in this case, like the analysis chip, generally designed as a disposable part and is also designated as a disposable adapter system. For example, a vessel in the form of a cartidge or syringe containing the disinfection fluid can be fitted onto the analysis chip. This embodiment is particularly suitable for the self-test market.

According to a second preferred embodiment, which is suitable in particular for the professional sector, i.e. for physicians and hospitals, the disinfection device is provided as a separate unit into which the analysis chip can be placed for disinfection. While the analysis chip is in this case likewise preferably a disposable part, the disinfection device is a comparatively large unit which is intended for frequent use and with which, depending on its design, a plurality of analysis chips can be disinfected simultaneously.

According to an expedient embodiment, the disinfection device is integrated in a data readout unit provided for reading the analysis chip. The disinfection of the analysis chip can thus take place simultaneously with the data evaluation, thus saving time. Moreover, this combining of the evaluation with the disinfection of the analysis chip affords the possibility of using data concerning the infectious properties of the tested biological sample, and of the analysis chip coming into contact with it, in order to determine the suitable disposal route.

An object of an embodiment of the invention is also includes a disinfection and evaluation device. This disinfection and evaluation device is intended for treating an analysis chip of the above-described type and includes, in addition to a disinfection device of any desired type, an evaluation unit for reading or evaluating the analysis chip in a manner dependent on the latter's configuration, in particular by optic and/or electronic methods. During the evaluation and disinfection, the analysis chip is preferably held in a fixed position. The advantages mentioned in connection with the analytical and diagnostic instrument, insofar as these relate to the embodiment concerning a unit for the professional sector, apply by analogy also to the disinfection and evaluation device.

### BRIEF DESCRIPTION OF THE DRAWINGS

The present invention will become more fully understood from the detailed description of preferred embodiments given hereinbelow and the accompanying drawing, which is given by way of illustration only and thus is not limitative of the present invention, and wherein:

In the drawing, the single FIGURE is a diagrammatic representation of an analytical and diagnostic instrument for testing biological samples.

### DETAILED DESCRIPTION OF THE PREFERRED EMBODIMENTS

An analytical and diagnostic instrument 1 includes an analysis chip 2 and a disinfection device 3, which is designed in two parts in the illustrative embodiment. In the analysis chip 2, biosensors 5 in the form of a biomolecular matrix are applied to a carrier 4, each point of the matrix representing an individual molecule species (DNA, RNA, proteins, peptides, polysaccharides, sugars or other chemical structures). A biological sample to be tested (not shown here) can flow through a fluid channel 6 of the analysis chip 2 and thus comes into contact with the biosensors 5.

The analysis chip 2 can, for example, be provided for testing blood, serum, plasma, urine, sputum, cerebrospinal fluid, stools, mucus, cell smears, cells, tissue samples, and nutrient and culture media, or other fluids. When using the analysis chip 2, it must always be borne in mind that the latter is contaminated by the test sample, in particular by bacteria, viruses or fungi. The contamination affects all surfaces of the analysis chip 2 which come into contact with the sample to be tested. If the analysis chip 2 is not disinfected after use, it is therefore generally to be classified as infectious.

In the illustrative embodiment, the disinfection device 3 has a storage vessel 7 and a collecting vessel 8 for a disinfection fluid F. The storage vessel 7 can be mechanically connected to the collecting vessel 8 in a manner not shown, so that the disinfection device 3 is designed as a one-part component into which the analysis chip 2 is placed or clamped for disinfection. The storage vessel 7 is joined to an inlet opening 9, and the collecting container 8 to an outlet opening 10, of the analysis chip 2. Mounted in the storage vessel 7 there is a piston 11 with which the disinfection fluid F is forced through the fluid channel 6 of the analysis chip 2.

The collecting vessel 8 is dimensioned such that it takes up both the volume of the disinfection fluid F contained in the storage vessel 7 and also the volume of the biological material to be tested. As an alternative to the embodiment described above, in which the storage vessel 7 and the collecting vessel 8 form a single device, the collecting vessel 8 can also be independent of the storage vessel 7. The homogeneously mixed material contained in the collecting vessel 8 after the disinfection is not infectious. The disinfected analysis chip 2 can therefore be disposed of as household waste together with the disinfection device 3 designed as disposable adapter system.

In addition to the first embodiment described above (disinfection predominantly in the self-test market), the figure also shows the function of the second embodiment (disinfection in the professional sector) . In the latter case, the storage vessel 7 and the collecting vessel 8 are simply parts of a larger disinfection device 3 (not shown), where the volumes of the vessels 7, 8 are designed for a large number of disinfection processes. The volume of disinfection fluid F forced through the analysis chip 2 in each disinfection process is, for example, about 5 ml. Instead of a liquid disinfection agent F, it is possible, particularly in the professional sector, to also use a gaseous disinfection fluid, for example ethylene oxide. Likewise, it is possible to use a number of gaseous, liquid and/or solid reagents which form a disinfection agent only by reaction.

If the disinfection device 3 is designed as a unit intended for multiple use, the analysis chip 2 can also be read in the illustrated position. For this purpose, an electrical connection (not shown) on the analysis chip 2 and on the disinfection device 3 is provided and/or an optical evaluation of the analysis chip 2 is provided. A data line indicated by a broken line symbolizes an evaluation unit 12 in the FIGURE. The disinfection device 3 thus functions, together with the evaluation unit 12 integrated in it or connected to it, as a disinfection and evaluation device for treating the analysis chip 2 and in particular permits waste classification of the analysis chip 2.

Moving away from the embodiment shown in the FIGURE, the disinfection of the analysis chip 2 can be carried out by any desired methods, for example physical methods, in particular UV or X-ray radiation. If the disinfection process would influence or impair the information stored on the analysis chip 2, for example through chemical reactions, the disinfection of the analysis chip 2 preferably takes place directly after the completion of the reading process. A data evaluation optionally conducted after the reading process inside or outside the evaluation unit 12 can thus take place simultaneously with the disinfection of the analysis chip 2.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An analytical and diagnostic instrument, comprising:
an analysis chip for testing biological material, the analysis chip including a carrier and biosensor, an inlet opening and an outlet opening for disinfection fluid; and
a disinfection device for applying the disinfection fluid to the analysis chip, wherein the disinfection device is further for carrying out a waste classification of the analysis chip.

2. The analytical and diagnostic instrument as claimed in claim 1, wherein the disinfection device is designed as a disposable adapter system, connectable to the analysis chip.

3. The analytical and diagnostic instrument as claimed in claim 1, wherein the disinfection device is designed as a re-usable unit for the disinfection of at least one analysis chip.

4. The analytical and diagnostic instrument as claimed in claim 3, wherein the disinfection device is further for reading the analysis chip.

5. The analytical and diagnostic instrument as claimed in claim 1, wherein the disinfection device includes a storage vessel, for holding the disinfection fluid, and a separate collecting vessel.

6. A disinfection and evaluation device for treating an analysis chip including a carrier and biosensor for testing biological material, comprising:
a disinfection device for disinfection of the analysis chip, wherein the disinfection device, in cooperation with an evaluation unit for evaluating the analysis chip, is further for carrying out a waste classification of the analysis chip.

7. The disinfection and evaluation device as claimed in claim 6, wherein the analysis chip is held in a fixed position during evaluation and disinfection.

8. The analytical and diagnostic instrument as claimed in claim 2, wherein the disinfection device includes a storage vessel, for holding the disinfection fluid, and a separate collecting vessel.

9. The analytical and diagnostic instrument as claimed in claim 3, wherein the disinfection device includes a storage vessel, for holding the disinfection fluid, and a separate collecting vessel.

10. The analytical and diagnostic instrument as claimed in claim 4, wherein the disinfection device includes a storage vessel, for holding the disinfection fluid, and a separate collecting vessel.

11. An analytical and diagnostic instrument, comprising:
analysis means for testing biological material, the analysis means including a carrier and biosensor, an inlet opening and an outlet opening for disinfection fluid; and
means for applying the disinfection fluid to the analysis means, and for carrying out a waste classification of the analysis chip.

12. The analytical and diagnostic instrument as claimed in claim 11, wherein means for applying the disinfection is designed as a disposable adapter system, connectable to the analysis means.

13. The analytical and diagnostic instrument as claimed in claim 11, wherein the means for applying disinfection is designed as a re-usable unit for the disinfection of at least one analysis means.

14. The analytical and diagnostic instrument as claimed in claim 13, wherein the means for applying disinfection is further for reading the analysis means.

15. The analytical and diagnostic instrument as claimed in claim 11, wherein the means for applying disinfection includes a storage means for holding the disinfection fluid, and a separate means for collecting the disinfection fluid.

16. A disinfection and evaluation device for treating an analysis chip including a carrier and biosensor for testing biological material, comprising:
means for disinfecting the analysis chip, wherein the means for disinfecting, in cooperation with means for evaluating the analysis chip, is further for carrying out a waste classification of the analysis chip.

17. The disinfection and evaluation device as claimed in claim 16, wherein the analysis chip is held in a fixed position during evaluation and disinfection.

18. An analytical and diagnostic instrument, comprising:
an analysis chip, adapted to test biological material, the analysis chip including a carrier and biosensor, an inlet opening and an outlet opening for disinfection fluid; and
a disinfection device, adapted to apply the disinfection fluid to the analysis chip, wherein the disinfection device is further adapted to carry out a waste classification of the analysis chip.

19. The analytical and diagnostic instrument as claimed in claim 18, wherein the disinfection device is designed as a disposable adapter system, connectable to the analysis chip.

20. A disinfection and evaluation device for treating an analysis chip including a carrier and biosensor for testing biological material, comprising:
a disinfection device, adapted to disinfect the analysis chip, wherein the disinfection device, in cooperation with an evaluation unit for evaluating the analysis chip, is further adapted to carry out a waste classification of the analysis chip.
